(19) United States
(12) Patent Application Publication
(10) Pub. No.: US 2023/0038069 A1
(43) Pub. Date: Feb. 9, 2023

(54) BALLOON-TYPE ELECTRODE CATHETER

(71) Applicant: Japan Lifeline Co., Ltd., Shinagawa-ku, Tokyo (JP)

(72) Inventors: Kenji MORI, Shinagawa-ku, Tokyo (JP); Toshiyuki IIJIMA, Shinagawa-ku, Tokyo (JP)

(21) Appl. No.: 17/786,973

(22) PCT Filed: Dec. 24, 2019
(86) PCT No.: PCT/JP2019/050724

§ 371 Date: Jun. 17, 2022

(51) Int. Cl. A61B 18/14

(52) U.S. Cl. CPC A61B 18/1492; A61B 2018/00791

(57) ABSTRACT

A balloon-type electrode catheter according to the present invention includes an outer tube, an energizing connector, a balloon including neck portions on both ends of an expansion portion, an inner tube, a distal end tip, strip electrodes formed on an outer surface of the balloon, a metal ring attached to the distal end side neck portion with a distal end portion of each of the strip electrodes secured to an outer circumferential surface of the metal ring, and a lead wire electrically connecting each of the strip electrodes and the energizing connector with a distal end of the lead wire fixed to an inner circumferential surface of the metal ring and with a proximal end of the lead wire fixed to the energizing connector.

### TECHNICAL FIELD

The present invention relates to a balloon-type electrode catheter, and more particularly to a balloon-type electrode catheter that is intravascularly introduced for high-frequency ablation treatment on a vessel or tissue surrounding the vessel.

### BACKGROUND ART

A known balloon-type electrode catheter (intravascular ablation apparatus) for high-frequency ablation treatment on a vessel or tissue surrounding the vessel includes an outer tube (catheter shaft), a balloon connected to a distal end of the outer tube, an inner tube (guide wire lumen) inserted inside a lumen of the outer tube and the balloon, a lumen tube (supply lumen) inserted into the lumen of the outer tube to supply a fluid to the inside of the balloon, a lumen tube (return lumen) inserted into the lumen of the outer tube to discharge the fluid supplied to the inside of the balloon, and a surface electrode (high-frequency electrode) provided on an outer surface of the balloon (see Patent Literature 1 below).

The balloon included in the balloon-type electrode catheter described in Patent Literature 1 includes an expansion portion that expands and contracts, and neck portions formed at both ends of the expansion portion. The proximal end side neck portion is fixed to the outer tube, and the distal end side neck portion is fixed to the inner tube (guide wire lumen).

With the balloon-type electrode catheter described in Patent Literature 1, high-frequency ablation treatment is performed on a vessel or tissue lesion surrounding the vessel by application of a high-frequency current to the surface electrode provided on the outer surface of the balloon. The inside of the balloon can be cooled with the fluid supplied to the inside of the balloon from the lumen tube (supply lumen) to be circulated inside the balloon and then discharged through the lumen tube (return lumen).

The inventor of the present invention has proposed, as a mode for applying a high-frequency current to a surface electrode formed on the outer surface of a balloon in a balloon-type electrode catheter for electrically isolating a pulmonary vein, a mode in which a metal ring is attached to the neck portion of the balloon fixed to the distal end portion of a catheter shaft (neck portion on the proximal end side on which an energizing connector is positioned), the surface electrode is electrically connected to this metal ring, and the metal ring and the energizing connector are electrically connected through a lead wire (see Patent Literature 2 below).

### CITATION LIST

### Patent Literature

Patent Literature 1: JP 2013-532564 T Patent Literature 2: JP 2016-185296 A (FIG. 4 in particular)

### SUMMARY OF INVENTION

### Technical Problem

The mode as described in Patent Literature 2 may be employed for applying a high-frequency current to the surface electrode formed on the outer surface of the balloon in the balloon-type electrode catheter for high-frequency ablation treatment on a vessel or tissue surrounding the vessel as described in Patent Literature 1. Specifically, a metal ring may be attached to the neck portion (proximal end side neck portion) of the balloon fixed to the outer tube (catheter shaft) and the current may be applied through this metal ring.

The lumen of the outer tube included in the balloon-type electrode catheter as described in Patent Literature 1 incorporates the inner tube (guide wire lumen) for insertion of a guide wire (guide wire lumen) and the lumen tubes for circulation of a cooling fluid (supply lumen and return lumen), and thus has a significantly large outer diameter.

In particular, when ablation treatment on a tumor or the like is performed with the balloon-type electrode catheter, the flow rate of the fluid circulating inside the balloon needs to be high in order to enhance the cooling effect of the tissue around the surface electrode. In view of this, the diameter of the lumen tube for circulation of a cooling fluid is required to be increased, and the outer diameter of the outer tube needs to be further increased accordingly.

In the case where the proximal end side neck portion of the balloon is fixed to the outer tube having such a large outer diameter and a metal ring is attached to the proximal end side neck portion, the outer diameter of the metal ring largely exceeds the outer diameter limited by a sheath and an endoscope (shaft diameter or wrapping diameter) used for introducing an electrode catheter. Thus, the metal ring gets caught in the openings of the sheath and the endoscope used for introducing the balloon-type electrode catheter, and the balloon-type electrode catheter fails to be inserted inside these lumens.

The present invention has been made based on the above-described circumstances. An object of the present invention is to provide a balloon-type electrode catheter that can be intravascularly introduced without compromising ease of insertion into the lumens of a sheath and an endoscope used and that can perform ablation treatment over a wide range on a vessel or tissue lesion surrounding the vessel. Another object of the present invention is to provide a balloon-type electrode catheter that can perform uniform ablation treatment on a vessel or tissue surrounding the vessel along the circumferential direction of the vessel. Still another object of the present invention is to provide a balloon-type electrode catheter that is excellent in the cooling effect inside the balloon and is thus excellent in the cooling effect on tissue around a surface electrode.

### Solution to Problem

(1) A balloon-type electrode catheter according to the present invention is a balloon-type electrode catheter intravascularly introduced for high-frequency ablation treatment on a vessel or tissue surrounding the vessel and includes:
   an outer tube including a central lumen and a plurality of sub-lumens disposed to surround the central lumen;
   an energizing connector disposed on a proximal end side of the outer tube;
   a balloon including an expansion portion that expands and contracts and continuous neck portions on both ends of the expansion portion, the balloon being connected on a distal end side of the outer tube with a proximal end side neck portion fixed to a distal end portion of the outer tube;
   an inner tube including a lumen allowing insertion of a guide wire therethrough, the inner tube being inserted into the central lumen of the outer tube, extending out inside the balloon through an opening of the central lumen, and extending inside the balloon;
   a distal end tip including a lumen that is in communication with the lumen of the inner tube, the distal end tip being connected to a distal end of the inner tube inside the balloon and fixed to a distal end side neck portion and extending outside the balloon;
   a surface electrode formed of a metal thin film formed on an outer surface of the balloon at the expansion portion and the distal end side neck portion;
   a metal ring attached to the distal end side neck portion of the balloon, the metal ring being electrically connected to the surface electrode with a distal end portion of the surface electrode secured to an outer circumferential surface of the metal ring; and
   a lead wire electrically connecting the surface electrode and the energizing connector with a distal end of the lead wire fixed to an inner circumferential surface of the metal ring, the lead wire extending inside the balloon and through any of the sub-lumens of the outer tube, and with a proximal end of the lead wire fixed to the energizing connector.

With the balloon-type electrode catheter having such a configuration, the surface electrode formed on the outer surface of the balloon can be electrically connected to the energizing connector through the metal ring and the lead wire, whereby a high-frequency current can be reliably applied to the surface electrode. With this configuration, ablation treatment can be performed over a wide range on a vessel or tissue lesion surrounding the vessel.

Since the distal end side neck portion of the balloon, to which the metal ring is attached, is a neck portion fixed to the distal end tip and has an outer diameter that is much smaller than that of the proximal end side neck portion fixed to the outer tube, the outer diameter of the metal ring attached to the distal end side neck portion can be made smaller than the outer diameters of the outer tube and the proximal end side neck portion. With this configuration, the metal ring is prevented from getting caught in the openings of the sheath and the endoscope used for introducing the balloon-type electrode catheter, and ease of insertion of the balloon-type electrode catheter into the lumens of the sheath and the endoscope is not compromised.
(2) Preferably in the balloon-type electrode catheter according to the present invention, the surface electrode includes a plurality of strip electrodes formed to extend along an axial direction of the balloon and disposed at equal angular intervals along a circumferential direction of the balloon, and a distal end portion of each of the strip electrodes is secured to the outer circumferential surface of the metal ring.

With the balloon-type electrode catheter having such a configuration, each of the plurality of strip electrodes formed at equal angular intervals along the circumferential direction of the balloon can be electrically connected to the energizing connector through the metal ring and the lead wire. Thus, a high-frequency current can be applied evenly to each of the plurality of strip electrodes, whereby uniform ablation treatment can be performed on a vessel or tissue surrounding the vessel along the circumferential direction of the vessel.
(3) Preferably in the balloon-type electrode catheter according to the present invention, the metal ring is coated for insulation.

With the balloon-type electrode catheter having such a configuration, a temperature increase in the metal ring can be prevented while a current is applied thereto, and ablation on normal tissue around the metal ring can be avoided.
(4) Preferably in the balloon-type electrode catheter according to the present invention, at least one of the sub-lumens included in the outer tube is a fluid supply sub-lumen that circulates a fluid to supply the fluid to the inside of the balloon, and
   at least one of the sub-lumens included in the outer tube is a fluid discharge sub-lumen that circulates the fluid supplied to the inside of the balloon to discharge the fluid from the inside of the balloon.

(5) Preferably in the balloon-type electrode catheter in (4) above, the distal end portion of the outer tube fixed to the proximal end side neck portion of the balloon is incorporated in the expansion portion of the balloon,
   an opening of the fluid supply sub-lumen is positioned on a distal end side relative to an intermediate position in an axial direction of the expansion portion, and
   an opening of the fluid discharge sub-lumen is positioned at or near the proximal end of the expansion portion.

With the balloon-type electrode catheter having such a configuration, since the positions of a supply port for a fluid into the inside of the balloon and a discharge port for the fluid from the inside of the balloon are shifted in the axial direction, the flow of the fluid from the distal end side to the proximal end side is formed even after the expansion of the balloon (after being filled with the fluid) to enable the fluid to flow inside the balloon. Therefore, the inside of the balloon and thus the tissue around the surface electrode can be cooled sufficiently.
(6) Preferably in the balloon-type electrode catheter in (4) and (5) above, the number of the fluid supply sub-lumen is greater than the number of the fluid discharge sub-lumen.

With the balloon-type electrode catheter having such a configuration as described above, the inside of the balloon can be maintained at a constant pressure (expansion pressure).
(7) Preferably in the balloon-type electrode catheter according to the present invention, an outer diameter of the distal end portion of the outer tube to which the proximal end side neck portion of the balloon is fixed is formed smaller than an outer diameter of the proximal end portion of the outer tube, and
   an outer diameter of the proximal end side neck portion of the balloon and the outer diameter of the proximal end portion of the outer tube are substantially equal.

With the balloon-type electrode catheter having such a configuration, since the outer diameter of the proximal end side neck portion having a maximum outer diameter is substantially equal to the outer diameter of the proximal end portion of the outer tube, ease of insertion into the lumens of the sheath and the endoscope is not compromised by the proximal end side neck portion. In addition, since the outer diameter of the outer tube can be the maximum diameter limited by the sheath and the endoscope, the diameters of the fluid supply sub-lumen and the fluid discharge sub-lumen included in the outer tube can be sufficiently ensured, whereby the cooling effect inside the balloon can be further improved.
(8) Preferably in the balloon-type electrode catheter according to the present invention, a temperature sensor is disposed on a tube wall of the balloon.

### Advantageous Effects of Invention

The balloon-type electrode catheter according to the present invention can be intravascularly introduced without compromising ease of insertion into the lumens of a sheath or an endoscope used and can perform ablation treatment over a wide range on a vessel or tissue lesion surrounding the vessel. In addition, the balloon-type electrode catheter according to the present invention including the surface electrode formed of the plurality of strip electrodes can perform uniform ablation treatment on a vessel or tissue surrounding the vessel along the circumferential direction of the vessel. Furthermore, the balloon-type electrode catheter according to the present invention including the outer tube that includes the fluid supply sub-lumen that is open on the distal end side relative to the intermediate position in the axial direction of the expansion portion of the balloon, and the fluid discharge sub-lumen that is open at or near the proximal end of the expansion portion of the balloon is excellent in the cooling effect inside the balloon and is thus excellent in the cooling effect on tissue around the surface electrode, compared with a known balloon-type electrode catheter.

### BRIEF DESCRIPTION OF DRAWINGS

FIG. 1 is a plan view of a balloon-type electrode catheter according to one embodiment of the present invention.

FIG. 2 is a partial cut-away front view of the balloon-type electrode catheter illustrated in FIG. 1 (front view including II-II cross section in FIG. 1).

FIG. 3 is a perspective view illustrating a distal end portion of the balloon-type electrode catheter illustrated in FIG. 1.

FIG. 4 is a perspective view illustrating the distal end portion (distal end side of the balloon) of the balloon-type electrode catheter illustrated in FIG. 1.

FIG. 5 is a perspective view illustrating a distal end portion (proximal end side of the balloon) of the balloon-type electrode catheter illustrated in FIG. 1.

FIG. 6 is a partially enlarged view (detailed view of VI part) of FIG. 2.

FIG. 7 is a partially enlarged view (detailed view of VII part) of FIG. 6.

FIG. 8 is a partially enlarged view (detailed view of VIII part) of FIG. 2.

FIG. 9 is a cross-sectional view taken along IX-IX in FIG. 1.

FIG. 10 is a partially enlarged view (detailed view of X part) of FIG. 9.

FIG. 11 is a cross-sectional view taken along XI-XI in FIG. 1.

FIG. 12 is a cross-sectional view taken along XII-XII in FIG. 1.

FIG. 13 is a partially enlarged view (detailed view of XIII part) of FIG. 12.

FIG. 14 is a cross-sectional view taken along XIV-XIV in FIG. 1.

FIG. 15 is a cross-sectional view taken along XV-XV in FIG. 1.

FIG. 16 is a partially enlarged view (detailed view of XVI part) of FIG. 15.

FIG. 17 is a cross-sectional view taken along XVII-XVII in FIG. 1.

FIG. 18 is a partially enlarged view (detailed view of XVIII part) of FIG. 17.

FIG. 19 is a cross-sectional view taken along XIX-XIX in FIG. 1.

FIG. 20 is a partially enlarged view (detailed view of XX part) of FIG. 19.

FIG. 21 is a cross-sectional view taken along XXI-XXI in FIG. 1.

FIG. 22 is a partially enlarged view (detailed view of XXII part) of FIG. 21.

FIG. 23 is a cross-sectional view taken along XXIII-XXIII in FIG. 1.

FIG. 24 is a cross-sectional view taken along XXIV-XXIV in FIG. 1.

### DESCRIPTION OF EMBODIMENTS

### Embodiments

A balloon-type electrode catheter 100 according to the present embodiment is a balloon-type electrode catheter intravascularly introduced for high-frequency ablation on a vessel or tissue lesion surrounding the vessel such as a tumor.

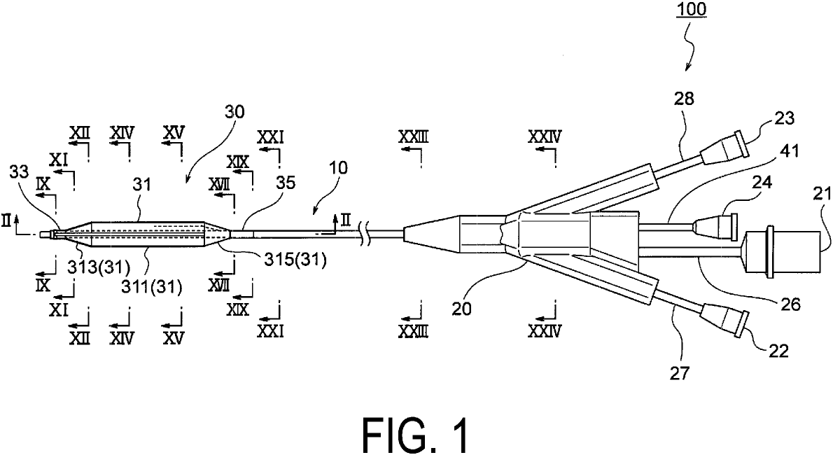

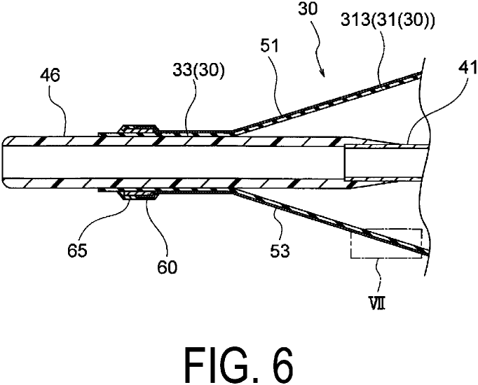

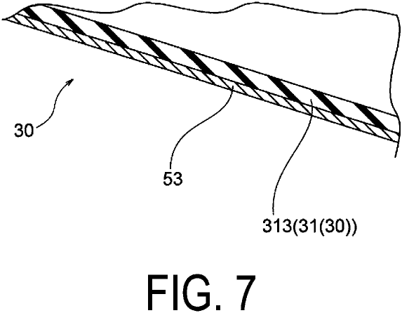

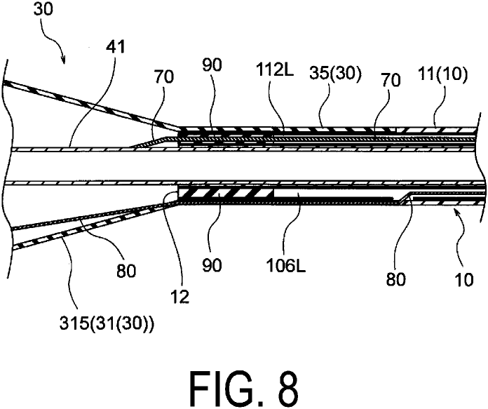

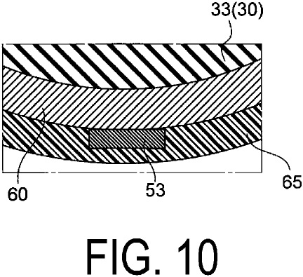

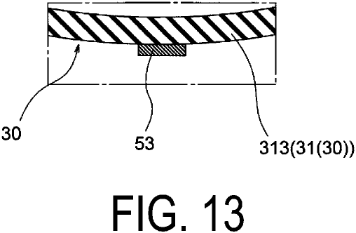

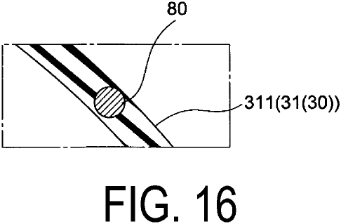

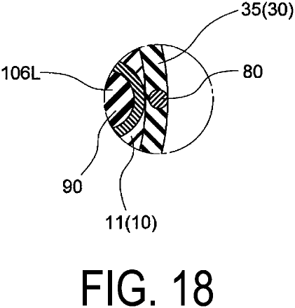

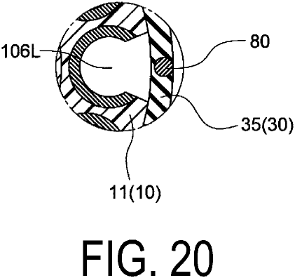

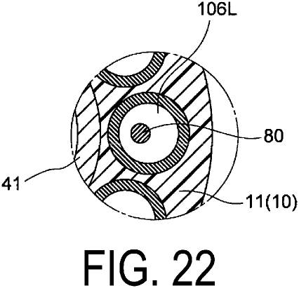

The balloon-type electrode catheter 100 illustrated in FIGS. 1 to 24 includes a circular tube portion 11 and a semicircular tube portion 13. The balloon-type electrode catheter 100 includes: an outer tube 10 including a central lumen 10L and sub-lumens 101L to 112L disposed to surround the central lumen 10L; an electric connector 21 disposed on a proximal end side of the outer tube 10; a balloon 30 including an expansion portion 31 that expands and contracts and continuous neck portions (distal end side neck portion 33 and proximal end side neck portion 35) on both ends of the expansion portion 31, the balloon 30 being connected on a distal end side of the outer tube 10 with the proximal end side neck portion 35 fixed to the circular tube portion 11 constituting a distal end portion of the outer tube 10, and the expansion portion 31 incorporating the semicircular tube portion 13 constituting the distal end portion of the outer tube 10; an inner tube 41 having a guide wire lumen, the inner tube 41 being inserted into the central lumen 10L of the outer tube 10, extending inside the balloon 30 through an opening of the central lumen 10L, and extending inside the balloon 30; a distal end tip 46 having a lumen (guide wire lumen) that is in communication with the guide wire lumen of the inner tube 41, the distal end tip 46 being connected to a distal end of the inner tube 41 inside the balloon 30 and fixed to the distal end side neck portion 33 and extending outside the balloon 30; strip electrodes 51 to 54 (surface electrode) formed of a metal thin film formed on an outer surface of the expansion portion 31 and the distal end side neck portion 33 of the balloon 30; a metal ring 60 attached to the distal end side neck portion 33 of the balloon 30, the metal ring 60 being electrically connected to each of the strip electrodes 51 to 54 with a distal end portion of each of the strip electrodes 51 to 54 secured to an outer circumferential surface of the metal ring 60; a lead wire 70 with a distal end thereof connected to an inner circumferential surface of the metal ring 60, the lead wire 70 extending inside the balloon 30 and through a sub-lumen 112L of the outer tube 10 (circular tube portion 11), and with a proximal end thereof connected to the electric connector 21; and a temperature sensor (thermocouple) 80 with a distal end thereof (temperature measuring unit 81) embedded in a tube wall of the expansion portion 31 of the balloon 30, the temperature sensor 80 extending along tube walls of the expansion portion 31 and the proximal end side neck portion 35 and through a lumen 106L of the outer tube 10 (circular tube portion 11), and with a proximal end thereof connected to the electric connector 21.

In FIGS. 1 and 2, 20 denotes a Y connector connected to the proximal end side of the outer tube 10, 22 denotes a fluid supply connector, 23 denotes a fluid discharge connector, 24 denotes a guide wire connector, 26 denotes a lead wire protection tube, 27 denotes a fluid supply tube, and 28 denotes a fluid discharge tube.

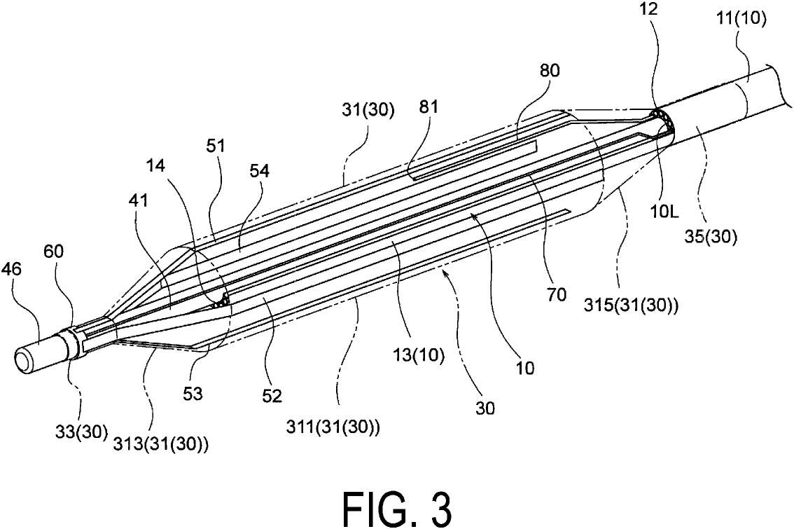

As illustrated in FIGS. 3 to 5, 14, 15, 17 to 22, the outer tube 10 included in the balloon-type electrode catheter 100 includes the circular tube portion 11 and the semicircular tube portion 13. The proximal end portion and the distal end portion of the outer tube 10 are formed partially by the circular tube portion 11, and the distal end portion of the outer tube 10 (excluding the portion mentioned above) is formed by the semicircular tube portion 13.

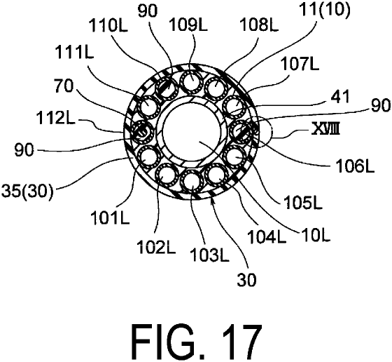

As illustrated in FIGS. 17, 19, and 21, inside the circular tube portion 11 of the outer tube 10, the central lumen 10L and twelve sub-lumens 101L to 112L disposed at equal angular (30°) intervals around the central lumen 10L are formed. In the circular tube portion 11, each of the sub-lumens 101L to 112L is formed of a lumen tube surrounding it. These lumen tubes are fixed by a binder resin that forms the circular tube portion 11.

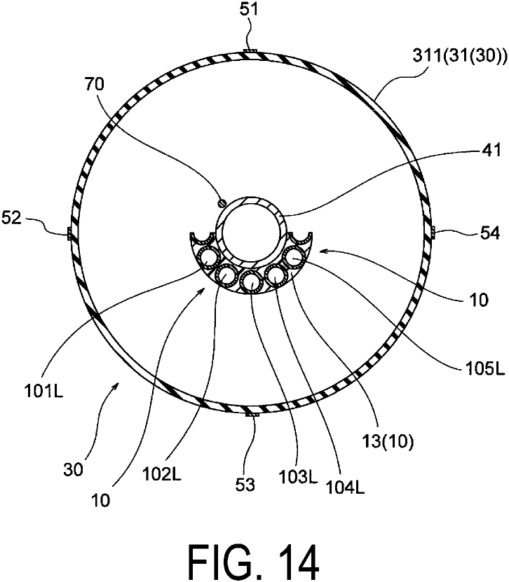

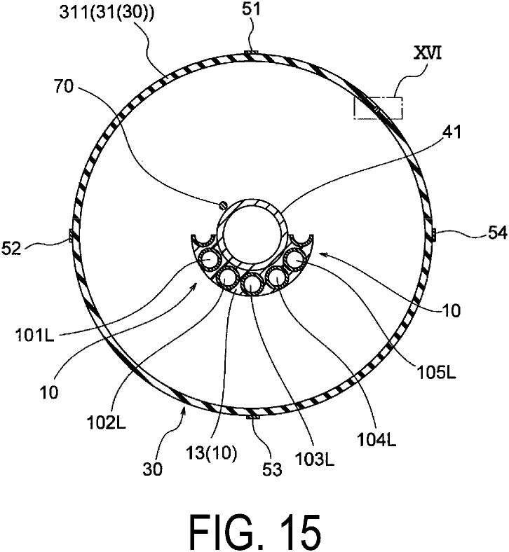

As illustrated in FIGS. 14 and 15, inside the semicircular tube portion 13 of the outer tube 10, the sub-lumens 101L to 105L are formed continuously from the inside of the circular tube portion 11. A lumen tube surrounding each of the sub-lumens 101L to 105L in the semicircular tube portion 13 is fixed by a binder resin that forms the semicircular tube portion 13.

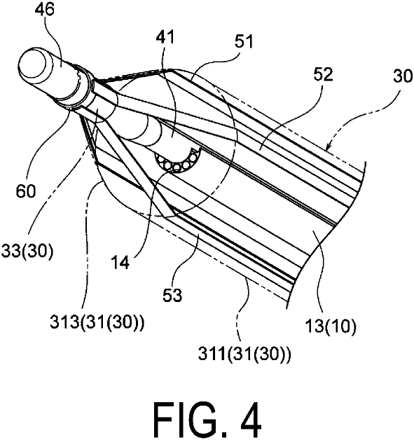

As illustrated in FIGS. 3 and 4, the sub-lumens 101L to 105L disposed inside the circular tube portion 11 and inside the semicircular tube portion 13 are each open at a distal end surface 14 of the semicircular tube portion 13, which is the distal end surface of the outer tube 10.

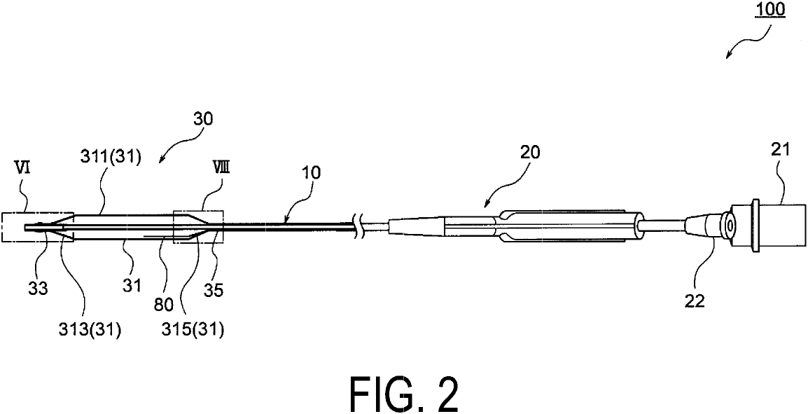

Each of the sub-lumens 101L to 105L is in communication with the fluid supply connector 22 illustrated in FIGS. 1 and 2. With this configuration, the sub-lumens 101L to 105L (five sub-lumens of the twelve sub-lumens formed in the outer tube 10) serve as “fluid supply sub-lumens” to supply a fluid to the inside of the balloon 30 (expansion portion 31). Here, examples of the fluid supplied to the inside of the balloon 30 can include saline.

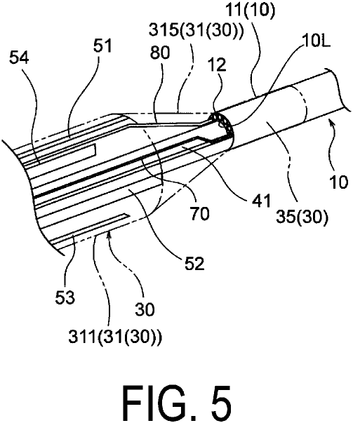

As illustrated in FIGS. 3 and 5, the central lumen 10L and the sub-lumens 106L to 112L formed inside the circular tube portion 11 are each open at a distal end surface 12 of the circular tube portion 11. Note that the openings of the sub-lumens 106L, 110L, and 112L are sealed by a sealing material 90 illustrated in FIG. 17.

Each of the sub-lumens 107L to 111L is in communication with the fluid discharge connector 23 illustrated in FIG. 1. With this configuration, the sub-lumens 107L to 109L and 111L (four sub-lumens of the twelve sub-lumens formed in the outer tube 10) serve as “fluid discharge sub-lumens” to discharge the fluid supplied to the inside of the balloon 30 (expansion portion 31) from the inside of the balloon 30.

The constituent material of the outer tube 10 is not limited to particular materials, and examples thereof include polyamide-based resins such as polyamide, polyether polyamide, polyether block amide (PEBAX (registered trademark)), and nylon. Among these, PEBAX is preferred.

The outer diameter of the outer tube 10 (the outer diameter at the proximal end portion described below) is typically from 1.0 to 3.3 mm, and is 1.45 mm as a preferable example. The diameter of the central lumen 10L of the outer tube 10 is typically from 0.35 to 0.95 mm, and is 0.85 mm as a preferable example. The diameter of the sub-lumens 101L to 112L of the outer tube 10 is typically from 0.10 to 0.75 mm, and is 0.25 mm as a preferable example. The length of the outer tube 10 is typically from 100 to 2200 mm, and is 1800 mm as a preferable example.

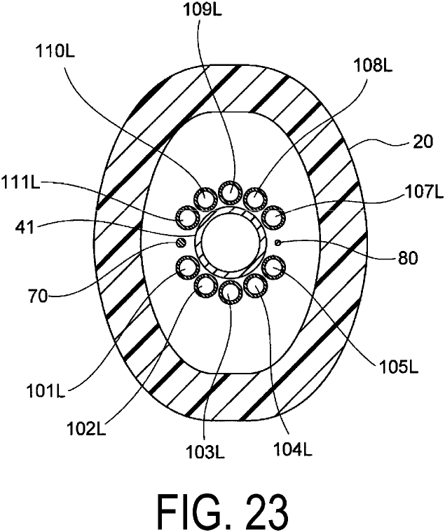

As illustrated in FIGS. 1 and 2, the Y connector 20 is connected to the proximal end side of the outer tube 10. As illustrated in FIG. 23, the lumen tubes surrounding the sub-lumens 101L to 105L and the sub-lumens 107L to 111L of the outer tube 10 enter the inside of the Y connector 20 from the proximal end of the outer tube 10.

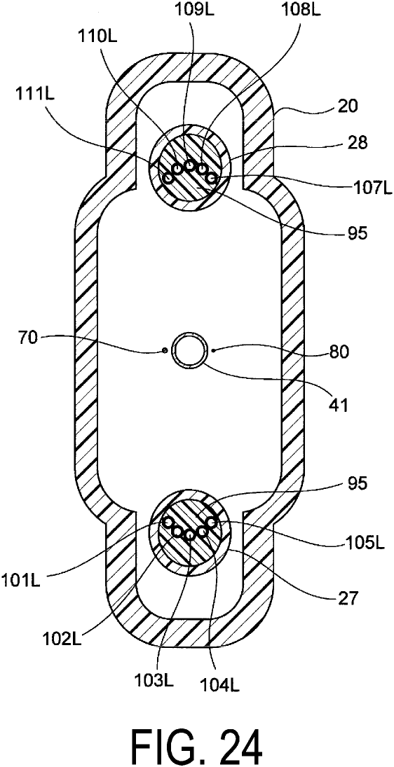

As illustrated in FIG. 24, the proximal end portions of the lumen tubes surrounding the sub-lumens 101L to 105L (fluid supply sub-lumen) are coupled (fixed with an adhesive 95) to the fluid supply tube 27 having a single lumen structure inside the Y connector 20. This fluid supply tube 27 extends outside the Y connector 20, and the proximal end of the fluid supply tube 27 is coupled to the fluid supply connector 22.

The proximal end portions of the lumen tubes surrounding the sub-lumens 107L to 111L are coupled (fixed with the adhesive 95) to the fluid discharge tube 28 having a single lumen structure inside the Y connector 20. This fluid discharge tube 28 extends outside the Y connector 20, and the proximal end of the fluid discharge tube 28 is coupled to the fluid discharge connector 23.

The balloon 30 included in the balloon-type electrode catheter 100 includes the expansion portion 31 that expands and contracts, the distal end side neck portion 33 that is continuous with the distal end of the expansion portion 31, and the proximal end side neck portion 35 that is continuous with the proximal end of the expansion portion 31.

The expansion portion 31 of the balloon 30 is a space-forming portion that expands with a fluid supplied to the inside thereof and contracts with the fluid discharged from the inside thereof. As illustrated in FIGS. 1 to 5, the expansion portion 31 of the balloon 30 includes a cylindrical portion 311, a distal end side cone portion 313 from the distal end of the cylindrical portion 311 to the proximal end of the distal end side neck portion 33, and a proximal end side cone portion 315 from the proximal end of the cylindrical portion 311 to the distal end of the proximal end side neck portion 35.

The balloon 30 is connected to the distal end side of the outer tube 10 with the distal end portion of the outer tube 10 (the distal end portion formed by the circular tube portion 11) fixed to the proximal end side neck portion 35 and with the distal end portion of the outer tube 10 (the distal end portion formed by the semicircular tube portion 13) incorporated in the expansion portion 31.

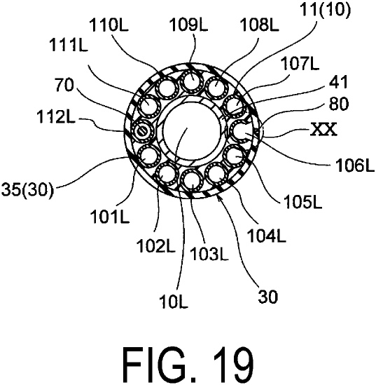

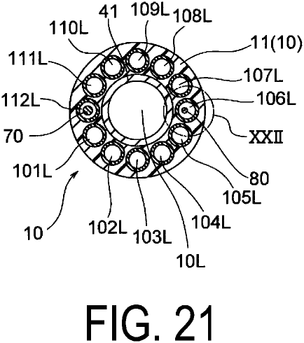

A surface layer portion of the distal end portion of the outer tube 10 to which the proximal end side neck portion 35 of the balloon 30 is fixed (the circular tube portion 11 illustrated in FIG. 19) is shaved off and has an outer diameter that is smaller than the outer diameter of the proximal end portion of the outer tube 10 to which the proximal end side neck portion 35 is not fixed (the circular tube portion 11 illustrated in FIG. 21). The outer diameter of the proximal end side neck portion 35 illustrated in FIG. 19 is substantially equal to the outer diameter of the proximal end portion of the outer tube 10 illustrated in FIG. 21.

With this configuration, ease of insertion into the lumens of the sheath and the endoscope used for introduction of the balloon-type electrode catheter 100 can be prevented from being compromised by the proximal end side neck portion 35. In addition, since the outer diameter of the outer tube 10 can be the maximum diameter limited by the sheath and the endoscope (no need for taking an increase in the outer diameter due to the thickness of the proximal end side neck portion into consideration), the diameters of the sub-lumens 101L to 112L of the outer tube 10 can be sufficiently ensured, whereby the cooling effect inside the balloon 30 can be further improved.

As illustrated in FIGS. 3 and 4, the distal end surface 14 of the semicircular tube portion 13, at which the fluid supply sub-lumens 101L to 105L are open, is positioned near the distal end of the cylindrical portion 311, which is on the distal end side relative to an intermediate position in an axial direction of the expansion portion 31 of the balloon 30. With this configuration, the fluid circulating through the fluid supply sub-lumens 101L to 105L is ejected in a direction toward the distal end from each of the openings positioned near the distal end of the cylindrical portion 311, and the ejected fluid can reach near the distal end of the expansion portion 31 (distal end side cone portion 313), whereby the flow of the fluid from the distal end side toward the proximal end side can be formed inside the balloon 30 (expansion portion 31).

If the opening positions of the fluid supply sub-lumens are on the proximal end side relative to the intermediate position in the axial direction of the expansion portion of the balloon, the fluid ejected from the openings in the direction toward the distal end fails to reach near the distal end of the expansion portion after the balloon has expanded, whereby the fluid flow from the distal end side to the proximal end side cannot be formed inside the balloon.

As illustrated in FIGS. 3 and 5, the distal end surface 12 of the circular tube portion 11 at which the fluid discharge sub-lumens 107L to 109L and 111L open is positioned at the proximal end of the expansion portion 31.

The constituent material of the balloon 30 is not limited to particular materials, and the materials of balloons included in known balloon catheters can be used. Examples thereof include polyamide-based resins such as polyamide, polyether polyamide, PEBAX, and nylon; and polyurethane-based resins such as thermoplastic polyether urethane, polyether polyurethane urea, fluorine polyether urethane urea, polyether polyurethane urea resin, and polyether polyurethane urea amide.

The diameter of the balloon 30 (expansion portion 31) is typically from 0.7 to 30.0 mm, and is 2.0 mm as a preferable example. The outer diameter of the proximal end side neck portion 35 of the balloon 30 is substantially equal to the outer diameter of the proximal end portion of the outer tube 10, is typically from 1.0 to 3.3 mm, and is 1.45 mm as a preferable example. The length of the balloon 30 (expansion portion 31) is typically from 8 to 50 mm, and is 20 mm as a preferable example.

In the balloon-type electrode catheter 100 according to the present embodiment, the inner tube 41 and the distal end tip 46 constitute an inner shaft. The inner tube 41 included in the balloon-type electrode catheter 100 has the lumen (guide wire lumen) allowing insertion of a guide wire therethrough. The inner tube 41 is inserted into the central lumen 10L of the outer tube 10 (circular tube portion 11) and has a distal end portion extending inside the balloon 30 (expansion portion 31) through the opening of the central lumen 10L.

The distal end portion of the inner tube 41 extending inside the balloon 30 (expansion portion 31) extends, with the semicircular portion on the outer circumferential surface covered by the semicircular tube portion 13, inside the proximal end side cone portion 315, the cylindrical portion 311, and the distal end side cone portion 313 of the expansion portion 31, and is coupled to the distal end tip 46 inside the distal end side cone portion 313.

Meanwhile, the proximal end portion of the inner tube 41 enters the inside of the Y connector 20 from the proximal end of the outer tube 10 (the opening on the proximal end side of the central lumen 10L) as illustrated in FIGS. 23 and 24, extends inside the Y connector 20, and extends outside the Y connector 20. The proximal end of the inner tube 41 is coupled to the guide wire connector 24.

As the constituent material of the inner tube 41, while the materials of inner tubes included in known balloon catheters can be used, PEEK resin (polyetheretherketone), which is a crystalline thermoplastic resin having excellent mechanical properties, is preferable.

The outer diameter of the inner tube 41 is the same as or slightly smaller than the diameter of the central lumen 10L of the outer tube 10 into which the inner tube 41 is inserted, and is typically from 0.34 to 0.99 mm, and is 0.84 mm as a preferable example. The inner diameter of the inner tube 41 is typically from 0.31 to 0.92 mm, and is 0.68 mm as a preferable example.

The distal end tip 46 included in the balloon-type electrode catheter 100 has the lumen (guide wire lumen) that is in communication with the guide wire lumen of the inner tube 41. The distal end tip 46 is connected to the distal end of the inner tube 41 inside the distal end side cone portion 313 of the expansion portion 31 of the balloon 30 and fixed to the distal end side neck portion 33 and extending outside the balloon 30. The distal end of the distal end tip 46 is open.

The constituent material of the distal end tip 46 is not limited to particular materials, and examples thereof include polyamide-based resins such as polyamide, polyether polyamide, PEBAX, and nylon; polyurethane, and the like.

The inner diameter of the distal end tip 46 is substantially the same as that of the inner tube 41, is typically from 0.31 to 0.92 mm, and is 0.68 mm as a preferable example. The outer diameter of the distal end tip 46 is typically from 0.35 to 2.6 mm, and is 1.0 mm as a preferable example. The outer diameter of the distal end side neck portion 33 of the balloon 30 to which the distal end tip 46 is fixed is typically from 0.37 to 3.3 mm, and is 1.18 mm as a preferable example.

As illustrated in FIGS. 3 to 7 and 9 to 15, as the surface electrode to which a high-frequency current is applied, the strip electrodes 51 to 54 formed of a metal thin film are disposed at 90° intervals along the circumferential direction of the balloon 30 so as to extend along the axial direction of the balloon 30, to the outer surface of the balloon 30 (the cylindrical portion 311 and the distal end side cone portion 313 of the expansion portion 31 and the distal end side neck portion 33).

Examples of the constituent material of the metal thin film constituting the strip electrodes 51 to 54 include gold, platinum, silver, copper, alloys of these, stainless steel, and the like. The film thickness of the metal thin film constituting the strip electrodes 51 to 54 is preferably from 0.5 to 5.0 µm, and more preferably from 1.0 to 2.5 µm. If this film thickness is excessively low, the temperature of the metal thin film may increase due to Joule heat during a procedure (during application of a high-frequency current). On the other hand, if the film thickness of the thin film is excessively great, the metal thin film is less likely to follow a change in the shape of the balloon according to expansion or contraction, and ease of expansion/contraction of the balloon may be compromised.

A method for forming the metal thin film constituting the strip electrodes 51 to 54 on the outer surface of the balloon 30 is not limited to a particular method. A typical method for forming a metal thin film, such as vapor deposition, sputtering, plating, printing, and the like, can be employed.

As illustrated in FIGS. 3, 4, 6, 9, and 10, the metal ring 60 is attached to the distal end side neck portion 33 of the balloon 30. The metal ring 60 included in the balloon-type electrode catheter 100 is fixed by caulking to the distal end side neck portion 33. The distal end portion of each of the strip electrodes 51 to 54 is secured to (brought into contact with) the outer circumferential surface of the metal ring 60. With this configuration, each of the strip electrodes 51 to 54 and the metal ring 60 are electrically connected.

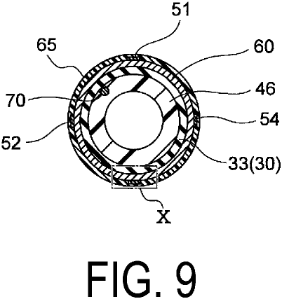

Examples of the constituent material of the metal ring 60 include platinum, platinum-based alloys, and the like. As illustrated in FIG. 9, the metal ring 60 is coated for insulation by a resin material 65 (not illustrated in FIGS. 3 and 4). With this configuration, a temperature increase in the metal ring 60 can be prevented while a current is applied thereto, and ablation on normal tissue around the metal ring 60 can be avoided.

The inner diameter of the metal ring 60 attached to the distal end side neck portion 33 is substantially the same as the outer diameter of the distal end side neck portion 33, is typically from 0.37 to 3.3 mm, and is 1.18 mm as a preferable example. The outer diameter of the metal ring 60 attached to the distal end side neck portion 33 is smaller than the outer diameters of the outer tube 10 and the proximal end side neck portion 35, is typically from 0.98 to 3.28 mm, and is 1.32 mm as a preferable example.

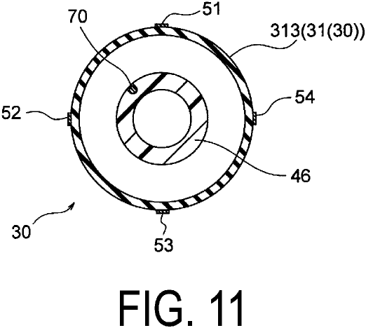

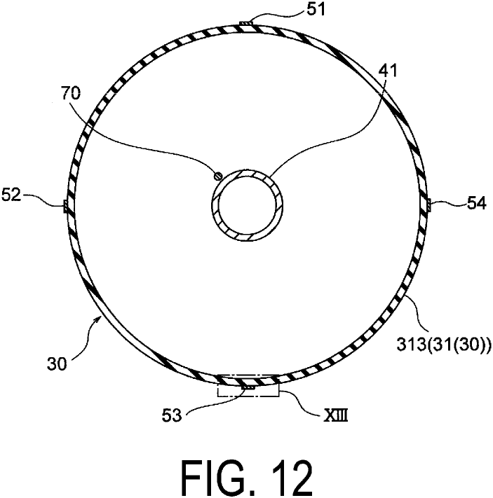

The distal end of the lead wire 70 is fixed to the inner circumferential surface of the metal ring 60. The lead wire 70 extends in the tube wall of the distal end tip 46 as illustrated in FIGS. 9 and 11, extends inside the expansion portion 31 of the balloon 30 along the inner tube 41 as illustrated in FIGS. 12, 14, and 15, extends through the sub-lumen 112L of the outer tube 10 (circular tube portion 11) as illustrated in FIGS. 17, 19, and 21, and extends inside the Y connector 20 and extends out of the Y connector 20 through the inside of the lead wire protection tube 26 extending from the Y connector 20 as illustrated in FIGS. 23 and 24.

The proximal end of the lead wire 70 is connected to the electric connector 21. The electric connector 21 functions as an energizing connector for applying a high-frequency current to each of the strip electrodes 51 to 54 and as a thermocouple connector for connecting a temperature sensor 80 to a temperature measuring instrument.

Connecting each of the strip electrodes 51 to 54 to the electric connector 21 through the metal ring 60 and the lead wire 70 can allow application of a high-frequency current evenly to each of the strip electrodes 51 to 54.

Examples of the constituent material of the lead wire 70 may include copper, silver, gold, platinum, tungsten, and alloys of these metals. The lead wire 70 is preferably provided with an electrically insulating protective coating such as a fluororesin.

As illustrated in FIGS. 3, 5, and 15 to 20, the temperature sensor 80 formed of a thermocouple is embedded and disposed in the tube wall of the balloon 30. The temperature measuring unit 81 (temperature measuring contact point) of the temperature sensor 80 is positioned in the tube wall of the expansion portion 31.

The temperature sensor 80 enters and extends through the sub-lumen 106L of the outer tube 10 (the circular tube portion 11) from the tube wall of the proximal end side neck portion 35 of the balloon 30 as illustrated in FIGS. 19 to 22, and together with the lead wire 70, extends inside the Y connector 20 and extends out of the Y connector 20 through the inside of the lead wire protection tube 26 extending from the Y connector 20 as illustrated in FIGS. 23 and 24. The proximal end of the temperature sensor 80 is connected to the electric connector 21.

With the balloon-type electrode catheter 100 according to the present embodiment, high-frequency ablation treatment can be performed over a wide range on a vessel or lesion surrounding the vessel by each of the strip electrodes 51 to 54 formed on the outer surface of the balloon 30.

Since the metal ring 60 is attached to the distal end side neck portion of the balloon 30 and the distal end portion of each of the strip electrodes 51 to 54 is secured to the outer circumferential surface of the metal ring 60, each of the strip electrodes 51 to 54 can be electrically connected to the electric connector 21 through the metal ring 60 and the lead wire 70. Thus, a high-frequency current can be applied evenly to each of the strip electrodes 51 to 54, whereby ablation treatment on a vessel or tissue lesion surrounding the vessel can be uniformly performed along the circumferential direction of the vessel.

The outer diameter of the metal ring 60 attached to the distal end side neck portion 33 of the balloon 30 is smaller than the outer diameters of the outer tube 10 and the proximal end side neck portion 35, which prevents the metal ring 60 from getting caught in the openings of the sheath and the endoscope used for introduction, and ease of insertion of the balloon-type electrode catheter 100 into the lumens of the sheath and the endoscope is not compromised.

Each of the fluid supply sub-lumens 101L to 105L is open at the distal end surface 14 of the semicircular tube portion 13 positioned near the distal end of the cylindrical portion 311 of the expansion portion 31 of the balloon 30. Each of the fluid discharge sub-lumens 107L to 109L and 111L is open at the distal end surface 12 of the circular tube portion 11 positioned at the proximal end of the expansion portion 31 of the balloon 30. Thus, the flow of the fluid from the distal end side to the proximal end side can be formed inside the balloon 30 even after the expansion of the balloon 30 (after being filled with the fluid), whereby the fluid can be flowed therein.

In particular, the fluid ejected in the direction toward the distal end from the openings of the fluid supply sub-lumens 101L to 105L hits the inner wall surface of the distal end side cone portion 313 of the expansion portion 31, and then flows in the direction toward the proximal end along the cylindrical portion 311 of the expansion portion 31 and the inner wall surface of the proximal end side cone portion 315, whereby the fluid can be circulated inside the balloon 30 (expansion portion 31).

As a result, the inside of the balloon 30 can be cooled efficiently over the entire area of the expansion portion 31, whereby the tissue around the strip electrodes 51 to 54 can be cooled sufficiently, and the fibrosing of the tissue can be prevented reliably.

Since the number of the fluid supply sub-lumens 101L to 105L is five and the number of the fluid discharge sub-lumens 107L to 109L and 111L is four, which are all disposed in the outer tube 10, the inside of the balloon 30 can be maintained at a constant pressure (expansion pressure).

Examples of the case to which the balloon-type electrode catheter 100 according to the present embodiment is applicable include a tumor or vagus nerve or the like on or surrounding a vessel, specific examples of which can include biliary cancer, lung cancer, liver cancer, kidney cancer, adrenal adenoma, renal artery vagus nerve, and the like.

While embodiments according to the present invention are described above, the present invention is not limited to these embodiments, and various modifications can be made. For example, the position of the distal end surface 14 of the semicircular tube portion 13 inside the balloon 30 (the opening positions of the fluid supply sub-lumens 101L to 105L) is not necessarily near the distal end of the cylindrical portion 311 of the expansion portion 31, as long as the position is on the distal end side relative to the intermediate position in the axial direction of the expansion portion 31.

In the balloon-type electrode catheter according to the present invention, the openings of the fluid supply sub-lumen and/or the fluid discharge sub-lumen may be formed on the outer circumferential surface of the outer tube, so that the fluid will be ejected/discharged in the radial direction of the outer tube.

The opening of the fluid supply sub-lumen may be positioned at or near the proximal end of the expansion portion of the balloon, whereas the opening of the fluid discharge sub-lumen may be positioned on the distal end side relative to the intermediate position in the axial direction of the expansion portion.

The fluid supply sub-lumen and the fluid discharge sub-lumen may be open at the same position in the axial direction. Furthermore, at least the portions of the strip electrodes 51 to 54 positioned in the distal end side cone portion 313 of the balloon 30 may be coated for insulation, so that ablation will be performed only with the portions of the strip electrodes 51 to 54 positioned in the cylindrical portion 311 of the balloon 30. With this configuration, restenosis in the tissue with which the distal end side cone portion 313 of the balloon 30 is brought into contact can be prevented. Here, a mode in which “at least the portions of the strip electrodes 51 to 54 positioned in the distal end side cone portion 313 of the balloon 30 are coated for insulation” can include a mode in which the entire areas of the distal end side cone portion 313 and the distal end side neck portion 33 are coated for insulation.

### REFERENCE SIGNS LIST

100 Balloon-type electrode catheter
10 Outer tube
10L Central lumen
101L to 105L Sub-lumen (fluid supply sub-lumen)
107L to 109L, 111L Sub-lumen (fluid discharge sub-lumen)
106L, 110L, 112L Sub-lumen
11 Circular tube portion
12 Distal end surface of circular tube portion
13 Semicircular tube portion
14 Distal end surface of semicircular tube portion
20 Y connector
21 Electric connector
22 Fluid supply connector
23 Fluid discharge connector
24 Guide wire connector
26 Lead wire protection tube
27 Fluid supply tube
28 Fluid discharge tube
30 Balloon
31 Expansion portion
311 Cylindrical portion
313 Distal end side cone portion
315 Proximal end side cone portion
33 Distal end side neck portion
35 Proximal end side neck portion
41 Inner tube
46 Distal end tip
51 to 54 Strip electrode (surface electrode)
60 Metal ring
70 Lead wire
80 Temperature sensor (thermocouple)
81 Temperature measuring unit of temperature sensor
90 Sealing material
95 Adhesive

1-8. (canceled)

9. A balloon-type electrode catheter comprising:
an outer tube;
a balloon including an expansion portion expanding and continuous neck portions on both ends of the expansion portion, a proximal end side neck portion fixed to a distal end portion of the outer tube;
a surface electrode formed of a metal formed on an outer surface of the balloon at the expansion portion and the distal end side neck portion;
a metal ring attached to the distal end side neck portion of the balloon, the metal ring being electrically connected to a distal end portion of the surface electrode; and
a lead wire with a distal end of the lead wire fixed to the metal ring, the lead wire extending inside the balloon and the outer tube to a proximal end portion of the outer tube, the lead wire being capable of being energized from the proximal end portion, wherein
an outer diameter of the metal ring is smaller than an outer diameter of the outer tube and the proximal end side neck portion.

10. The balloon-type electrode catheter according to claim 9, wherein
the outer tube includes a central lumen and a plurality of sub-lumens disposed to surround the central lumen,
the balloon-type electrode catheter comprises an inner tube including a lumen, the inner tube being inserted into the central lumen of the outer tube and extending inside the balloon, and the lead wire extends through any of the sub-lumens of the outer tube.

11. The balloon-type electrode catheter according to claim 10, comprising a distal end tip including a lumen in communication with the lumen of the inner tube, the distal end tip being fixed to the distal end side neck portion of the balloon.

12. The balloon-type electrode catheter according to claim 10, wherein the number of the sub-lumen at the proximal end portion of the outer tube is greater than the number of the sub-lumen at the distal end portion of the outer tube.

13. The balloon-type electrode catheter according to claim 10, wherein
at least one of the sub-lumens included in the outer tube is a fluid supply sub-lumen configured to supply a fluid to the inside of the balloon, and
at least one of the sub-lumens included in the outer tube is a fluid discharge sub-lumen configured to discharge the fluid supplied to the inside of the balloon from the inside of the balloon.

14. The balloon-type electrode catheter according to claim 13, wherein
the distal end portion of the outer tube fixed to the proximal end side neck portion of the balloon is incorporated in the expansion portion of the balloon,
an opening of the fluid supply sub-lumen is positioned on a distal end side relative to an intermediate position in an axial direction of the expansion portion, and
an opening of the fluid discharge sub-lumen is positioned on a proximal end side relative to the intermediate position in the axial direction of the expansion portion.

15. The balloon-type electrode catheter according to claim 13, wherein the number of the fluid supply sub-lumen is greater than the number of the fluid discharge sub-lumen.

16. The balloon-type electrode catheter according to claim 9, wherein
the surface electrode is a plurality of strip electrodes formed to extend along an axial direction of the balloon and disposed at intervals along a circumferential direction of the balloon, and
a distal end portion of each of the strip electrodes is electrically connected to the metal ring.

17. The balloon-type electrode catheter according to claim 9, wherein the metal ring is coated for insulation.

18. The balloon-type electrode catheter according to claim 17, wherein the distal end side neck portion is coated for insulation together with the metal ring.

19. The balloon-type electrode catheter according to claim 17, wherein
the expansion portion includes a cylindrical portion and a cone portion connecting the cylindrical portion and the neck portion, and
the cone portion on a distal end side is coated for insulation together with the metal ring.

20. The balloon-type electrode catheter according to claim 9, wherein an outer diameter of the distal end portion of the outer tube to which the proximal end side neck portion of the balloon is fixed is formed smaller than an outer diameter of the proximal end portion of the outer tube.

21. The balloon-type electrode catheter according to claim 9, wherein a temperature sensor is disposed on a tube wall of the balloon.
